(19) United States
(12) Patent Application Publication
(10) Pub. No.: US 2005/0055066 A1
(43) Pub. Date: Mar. 10, 2005

(54) Medium and low frequency electro-stimulated massaging bra

(76) Inventor: Tsung I. Yu, Taipei (TW)

Correspondence Address: BACON & THOMAS, PLLC, 625 SLATERS LANE, FOURTH FLOOR, ALEXANDRIA, VA, 22314, US

(21) Appl. No.: 10/658,188

(22) Filed: Sep. 10, 2003

(51) Int. Cl. A61N 1/18

(52) U.S. Cl. 607/48

(57) ABSTRACT

A medium and low frequency electro-stimulated massaging bra includes a plurality of conductive strips embedded within a bra on predetermined locations. Each of the conductive strips has a first end connected with a conductive plate and a second end connected with a conductive button. The conductive buttons are clustered on a predetermined location of the bra, which can be coupled with a corresponding set of conductive buttons on a electro-stimulating controller so as to mount the controller onto the bra. The electro-stimulating controller charges the conductive plates, which are in direct contact with a human chest, to produce the effect of a medium and low frequency electro-stimulated massage on the chest.

### BACKGROUND OF THE INVENTION

(a) Field of the Invention

The present invention relates to medium and low frequency electro-stimulated massaging devices, and more particularly to a medium and low frequency electro-stimulated massaging bra, which is provided with a clustered conductive button set for mounting an electro-stimulating controller thereon to achieve the effect of massage and fat reduction.

(b) Description of the Prior Art

The devices for medium and low frequency wave massaging in the prior art are mainly composed of a controller and electrodes made of silica gel (or conducting gel or electrode cups made of silica gel). Those devices are disadvantageous in high production cost and low electrical conductivity and can only be applied in special occasions. Especially, silica gel must be smeared over body portions the electro-stimulating massage is applied to, which inevitably causes discomfort and cleansing problem to a user. Furthermore, silica-gel plates and silica-gel cups are both hard objects, which are heavy and uncomfortable to wear, substantially reducing the portability thereof.

### SUMMARY OF THE INVENTION

The primary objective of the present invention is to provide a medium and low frequency electro-stimulated massaging bra having a plurality of conductive strips. Each of the conductive strips has a first end connected with a conductive plate and a second end connected with a conductive button. The conductive plates are in direct contact with a human chest when the bra is put on. The conductive buttons are clustered on a predetermined location of the bra, which can be coupled with a corresponding set of conductive buttons on a electro-stimulating controller so as to mount the controller onto the bra. The electro-stimulating controller charges the conductive plates to produce the effect of a medium and low frequency electro-stimulated massage on predetermined portions on the chest, which may enlarge the breasts, actuate a rehabilitating exercise, promote blood circulation and induce fat reduction.

The secondary objective of the present invention is to provide a medium and low frequency electro-stimulated massaging bra, wherein one or two control buttons for adjusting the strength of electro-stimulation can be installed on the lateral extended portions of the bra. The control buttons are connected with the conductive strips that converge to the conductive button set. After mounting the electro-stimulating controller onto the bra, the control buttons can be pressed to adjust the strength of electro-stimulation.

It is a further objective of the present invention that the medium and low frequency electro-stimulated massaging bra may have the electro-stimulating controller separable from the bra, utilizing a wire connection. Thereby, the electro-stimulating controller can be put in a clothes pocket or held in a hand for controlling the electro-stimulated massage.

The various objects and advantages of the present invention will be more readily understood from the following detailed description when read in conjunction with the appended drawings.

### BRIEF DESCRIPTION OF THE DRAWINGS

FIG. 1 is a perspective view of the bra according to the present invention when put on a human body.

FIG. 2 is a perspective view of the bra according to the present invention having an electro-stimulating controller mounted thereon.

FIG. 3 is a front perspective view of the electro-stimulating controller of the present invention.

FIG. 4 is a rear perspective view of the electro-stimulating controller of the present invention.

FIG. 5 is a cross-sectional side view of the electro-stimulating controller mounted on the bra.

FIG. 6 is an exploded view of the conductive plates, the conductive buttons and the cloth layers of the bra according to the present invention.

FIG. 7 is the circuit layout within the electro-stimulating controller of the present invention.

FIG. 8 is a perspective view of the present invention used with a cushion piece.

FIG. 9 is a side view of the present invention before a cushion piece is inserted therein.

FIG. 10 is a side view of the present invention after a cushion piece is inserted therein.

FIG. 11-FIG. 16 illustrate preferred embodiments of the present invention in which the conductive plates are deployed on a variety of locations.

FIG. 17 is a perspective view of a preferred embodiment of the present invention in which conductive plates and an electro-stimulating controller are combined to form an independent unit.

FIG. 18 shows independent unit composed of conductive plates and an electro-stimulating controller is applied within a bra.

FIG. 19 shows another preferred embodiment of the present invention in which the electro-stimulating controller is connected to the clustered conductive buttons of the bra by a wire.

### DETAILED DESCRIPTION OF THE PREFERRED EMBODIMENTS

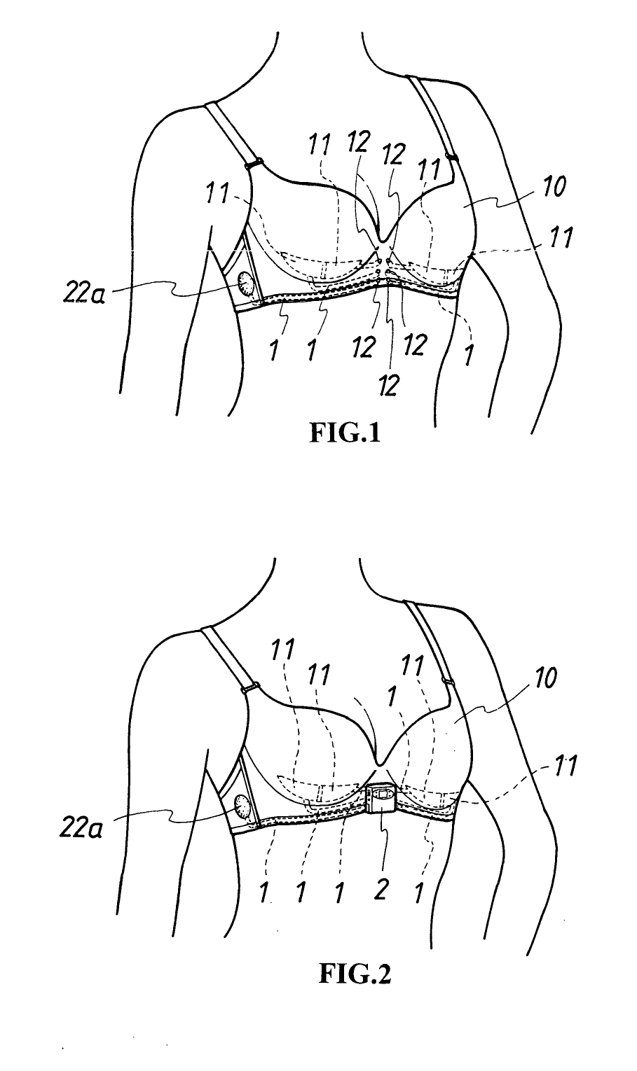

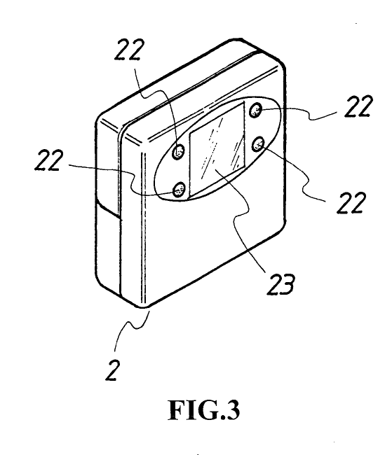

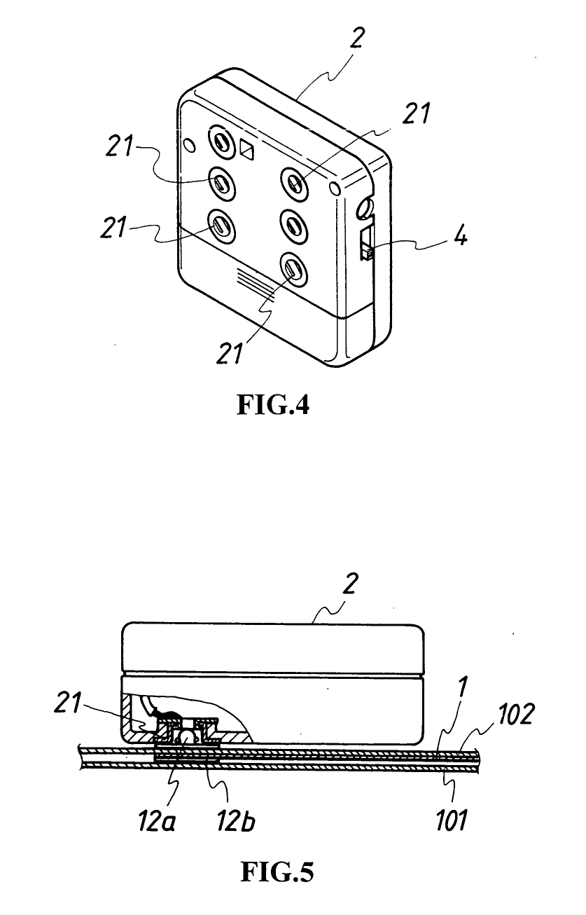

Referring to FIG. 1 and FIG. 2, a medium and low frequency electro-stimulated massaging bra according to the present invention is basically a bra 10 having a plurality of conductive strips 1 attached on selective locations thereon. Each of the conductive strips 1 has a first end connected with a conductive plate 11 and a second end connected with a conductive button 12. The conductive buttons 12 are clustered on a pre-determined location of the bra 10, which can be coupled with a corresponding set of conductive buttons 21 on a electro-stimulating controller 2 so as to mount the controller 2 onto the bra 10, as shown in FIG. 2 to FIG. 5. The electro-stimulating controller 2 charges the conductive plates 11, which are in direct contact with a human chest when the bra 10 is put thereon, to produce the effect of an medium and low frequency electro-stimulated massage.

The above-mentioned conductive strips 1 and conductive plates 11 are sewn on the bra 10, having the conductive strips 1 embedded in the inner layer of the bra and the conductive plates 11 uncovered for direct contact with a human chest.

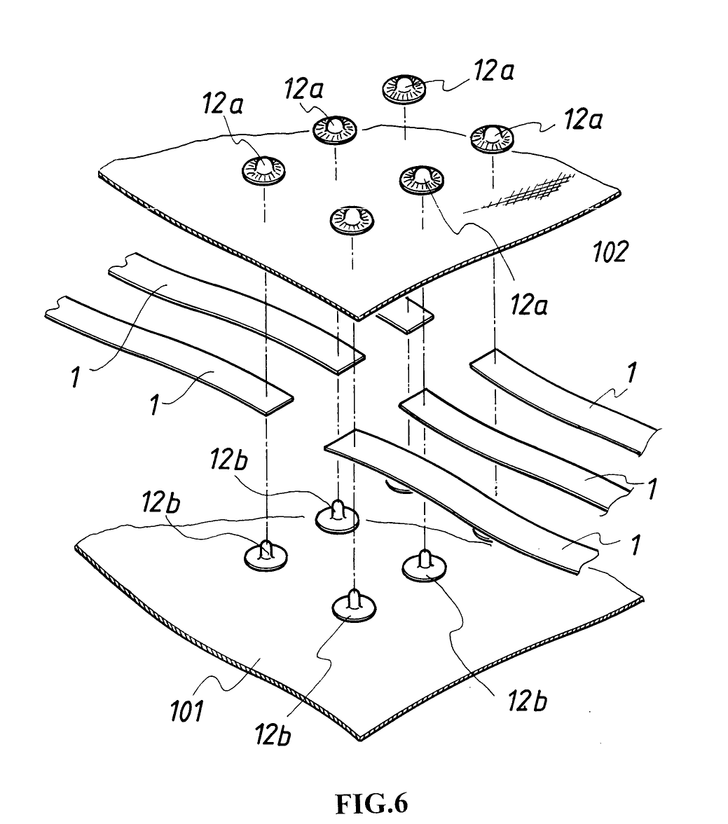

Each of the above-mentioned conductive strips 1 has the second end connected to a conductive button 12, which consists of a female piece 12a and a male piece 12b, respectively attached on an outer cloth layer 102 and an inner cloth layer 101. Referring to FIG. 6, as the female piece 12a and the male piece 12b are being rivet-connected, the associated conductive strip 1 is secured within the bra 10, whereas the female piece 12a extends outside the bra 10, providing a connection to a conductive button 21 on the electro-stimulating controller 2.

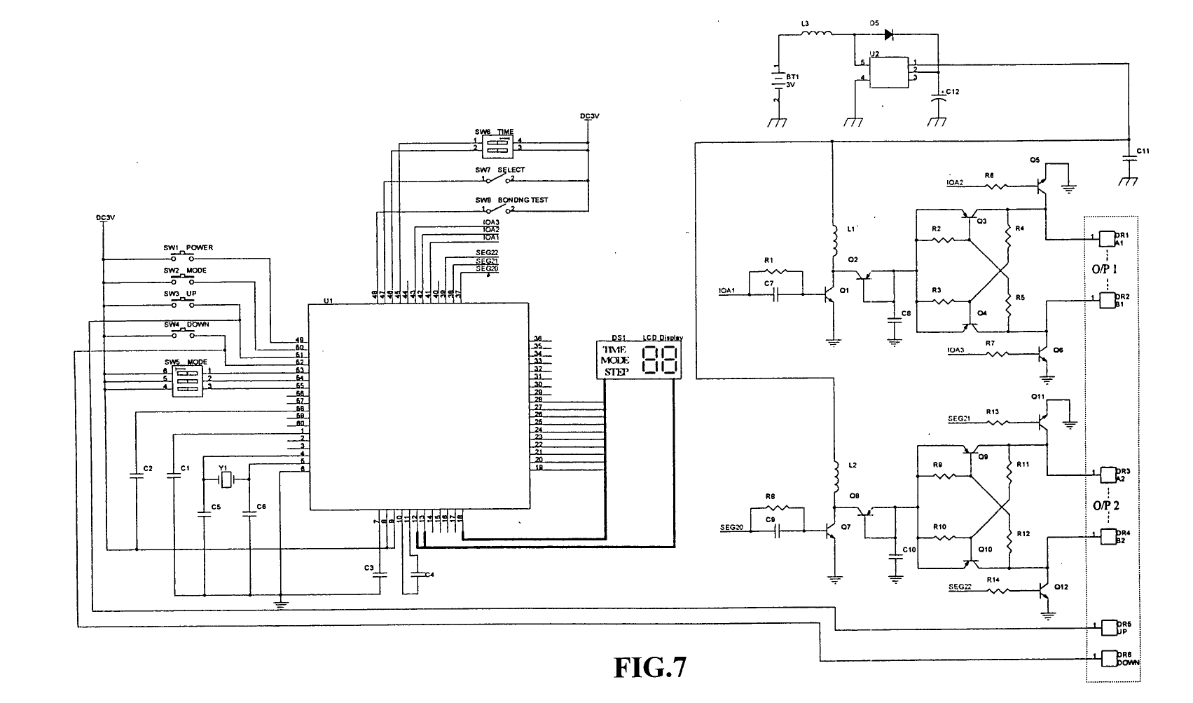

The electro-stimulating controller 2 is provided with a central integrated circuit (IC) and a charging/discharging circuit therein, as shown in FIG. 7. The IC transports a pulsed-wave signal to the circuit for controlling the charging/discharging of the capacitors and inductors thereon by which a high voltage is generated for providing an electro-stimulating effect. The IC further adjusts the bandwidth of the pulsed-wave signal, in a range from 1 Hz to 150 Hz, to produce massaging effect of various strengths. Furthermore, the electro-stimulating controller 2 has a plurality of control buttons 22 for respectively selecting current amplitude, operation time, power on/off and massage mode. The electro-stimulating controller 2 further contains an LCD display for displaying the operation status. The pulsed high voltage generated by the internal circuit (as shown in FIG. 7) is connected to a plurality of conductive buttons 21, which then form an output terminal. A stepping switch 24 is installed on a lateral side of the electro-stimulating controller 2 for selecting the charging region.

One or two control buttons 22a for adjusting the strength of electro-stimulation can be installed on the lateral extended portions of the bra 10. The control buttons 22a are connected with the conductive strips 1 that lead to where the conductive buttons 12 are clustered. After mounting the electro-stimulating controller 2 onto the bra 10, the control buttons 22a are pressed to adjust the strength of electro-stimulation.

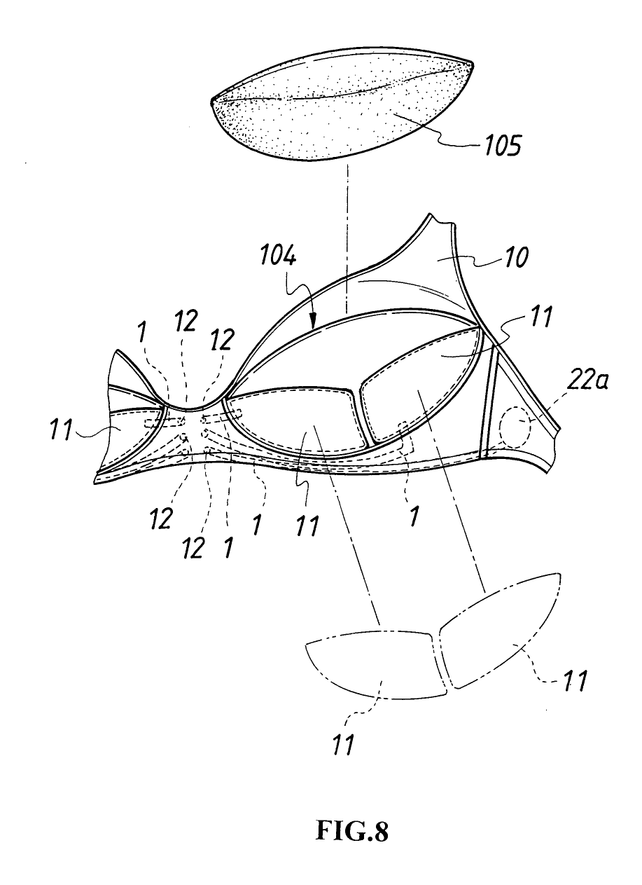

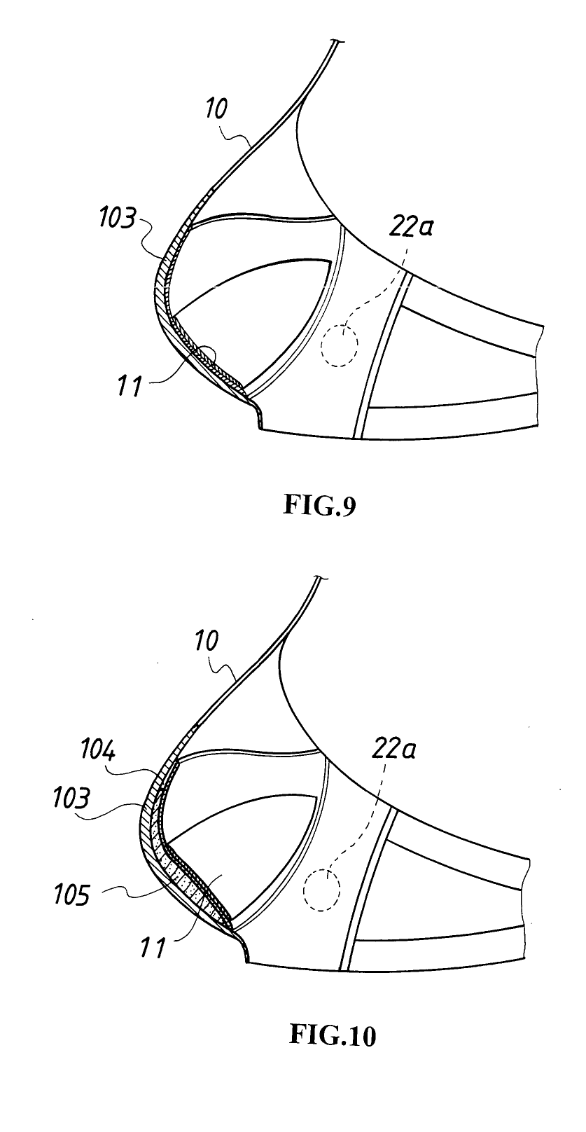

Referring to FIG. 8 and FIG. 9, a pocket 104 is formed on the bra 10 between each of the conductive plates 11 and the corresponding bra outer layer 103 for embedding a cushion piece 105 therein. The cushion pieces 105 push the respective conductive plates 11 further onto the human chest to enhance the contact, as shown in FIG. 10, which particularly suits people with a small breast size.

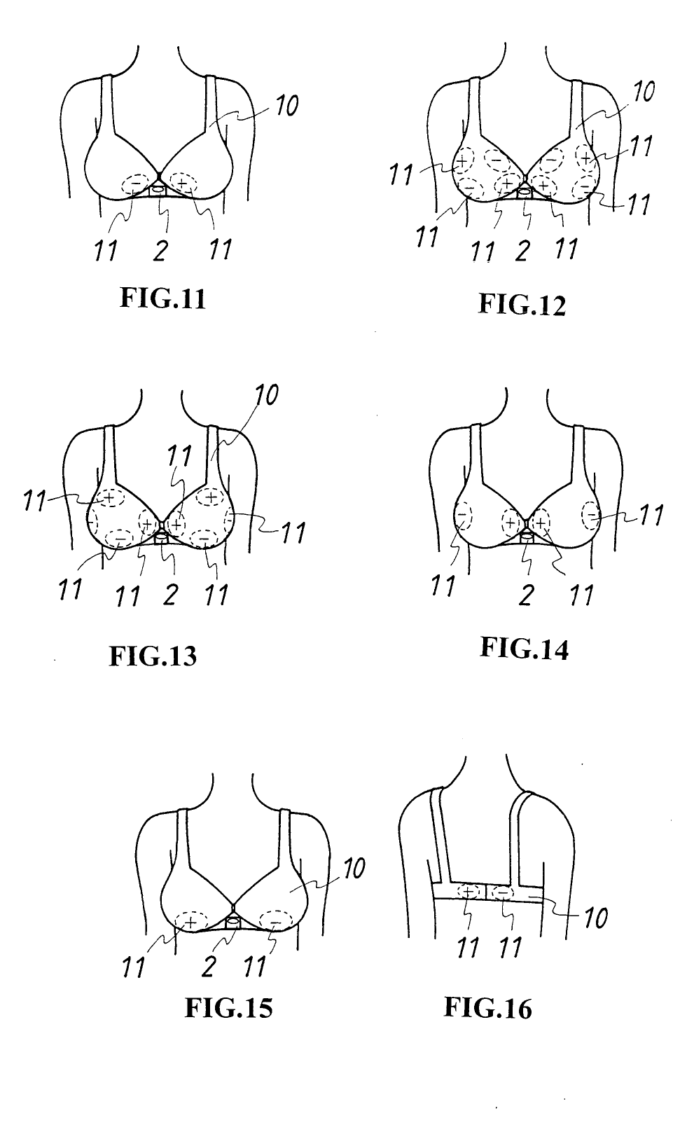

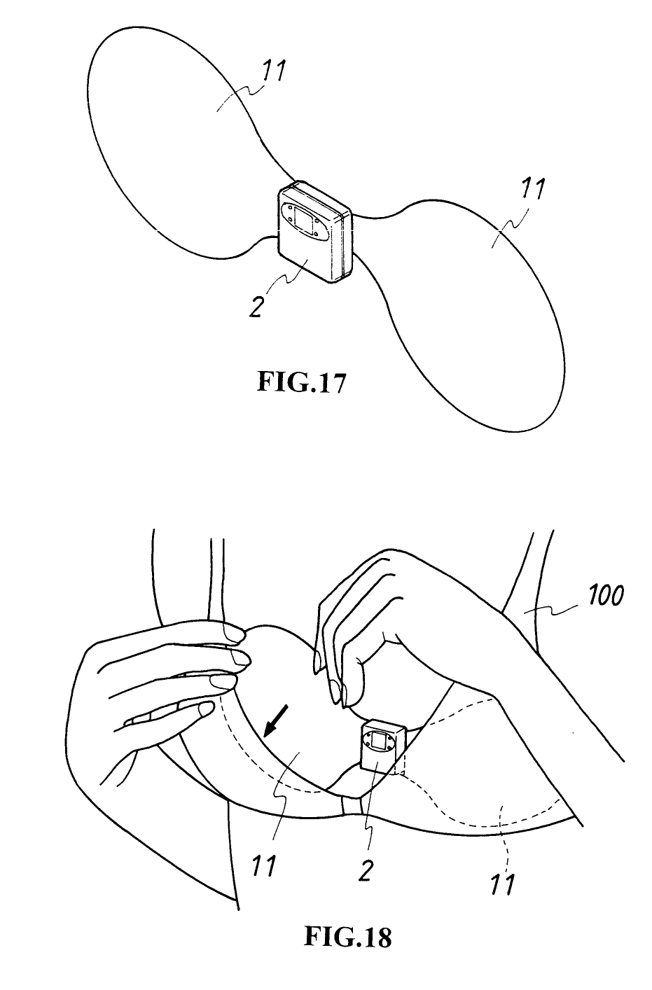

Referring to FIG. 11 to FIG. 16, the conductive strips 1 of the present invention can each be connected with more conductive plates 11. Depending on the designated function, the conductive plates 11 along a conductive strip 1 can be deployed on bra locations that corresponds to acupuncture points or fat-accumulative places of the human chest, so as to achieve functions such as enlarging breasts, rehabilitation or fat reduction.

Without departing from the spirit of the present invention, one or more than conductive plates 11 can be directly connected with an electro-stimulating controller 2 by installing conductive buttons 12 on the plates. Therefore, the conductive plates 11 and the electro-stimulating controller 2 form an independent unit that can be inserted into an ordinary bra 100 on a preferred location, having the conductive plates 11 directly contact with the human chest. The combined unit is then capable of exerting an electro-stimulated massage to the chest it applies to. In this preferred embodiment, a special bra in which conductive strips 1 are embedded is not necessary, greatly enhancing the applicability of the present invention.

The conductive strips 1 of the present invention are made of ordinary electro-conductive materials, and preferably a soft electro-conductive cloth.

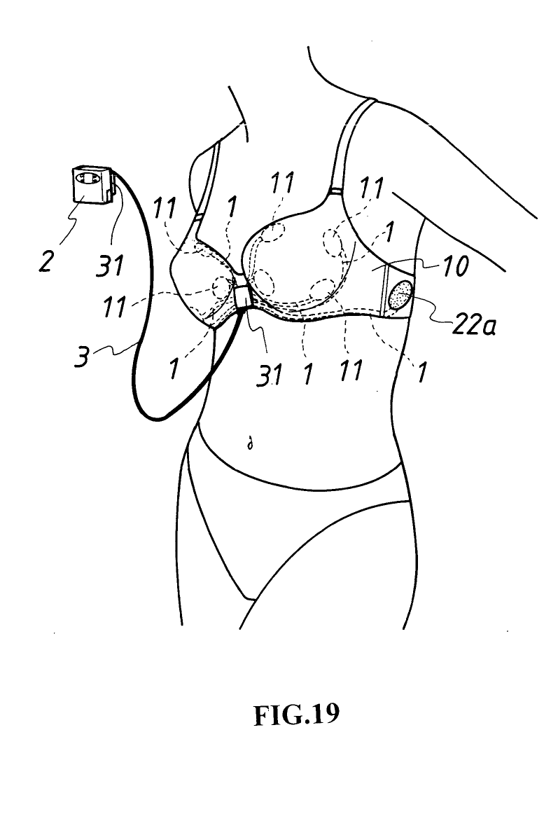

Referring to FIG. 19, the medium and low frequency electro-stimulated massaging bra according to the present invention may have the electro-stimulating controller 2 separable from the bra 10. A wire 3 is used to connect the electro-stimulating controller 2 and the bra 10. The wire has a first end connected with the clustered conductive buttons 12 on the bra 10 and a second end connected with the conductive buttons 21 on the electro-stimulating controller 2, by which the electro-stimulating controller 2 can be put in a clothes pocket or held in a hand for controlling the electro-stimulated massage.

The present invention is thus described, and it will be obvious that the same may be varied in many ways. Such variations are not to be regarded as a departure from the spirit and scope of the present invention, and all such modifications as would be obvious to one skilled in the art are intended to be included within the scope of the following claims.

1. A medium and low frequency electro-stimulated massaging bra comprising:
a bra;
a plurality of conductive plates attached on an inner side of said bra and in directly contact with a human chest when said bra is put on;
a plurality of conductive buttons clustered on a predetermined location of said bra;
a plurality of conductive strips, each having a first end connected to one of said conductive plates and a second end connected to one of said conductive buttons; and
an electro-stimulating controller having a corresponding set of conductive buttons for coupling with said conductive buttons of said bra, said electro-stimulating controller charging said conductive plates to produce an effect of medium and low frequency electro-stimulated massage.

2. The medium and low frequency electro-stimulated massaging bra of claim 1, wherein said conductive strips and said conductive plates are sewn on the bra; said conductive strips are embedded in an inner layer of said bra and said conductive plates are uncovered so as to provide direct contact with a human body.

3. The medium and low frequency electro-stimulated massaging bra of claim 1, wherein said electro-stimulating controller is provided with a central integrated circuit (IC) and a charging/discharging circuit; said IC is used for transporting a pulsed-wave signal to control the charging/discharging of a group of capacitors and inductors on said circuit; said IC further adjusts the bandwidth of said pulsed-wave signal so as to generate high voltages for providing medium and low frequency electro-stimulated massaging effects of various strengths.

4. The medium and low frequency electro-stimulated massaging bra of claim 3, wherein said bandwidth of said pulsed-wave signal is selected in a range from 1 Hz to 150 Hz.

5. The medium and low frequency electro-stimulated massaging bra of claim 1, wherein said pulsed high voltage generated by said electro-stimulating controller is connected to a plurality of conductive buttons thereon; said conductive buttons of said electro-stimulating controller then form an output terminal.

6. The medium and low frequency electro-stimulated massaging bra of claim 1, wherein a stepping switch is installed on a lateral side of said electro-stimulating controller for selecting a charging region.

7. The medium and low frequency electro-stimulated massaging bra of claim 1, wherein two control buttons for adjusting electro-stimulation strength are each installed on a lateral extended portion of said bra; said control buttons are connected with said conductive strips that lead to said clustered conductive buttons on which said electro-stimulating controller is mounted.

8. The medium and low frequency electro-stimulated massaging bra of claim 1, wherein said conductive buttons said conductive strips are connected with each consist of a female piece and a male piece; said female piece and said male piece are rivet-connected between an outer and an inner cloth layers of said bra.

9. The medium and low frequency electro-stimulated massaging bra of claim 1, wherein a pocket is formed on said bra between each of said conductive plates and a corresponding bra outer layer for embedding a cushion piece therein.

10. The medium and low frequency electro-stimulated massaging bra of claim 1, wherein said second end of each of said conductive strips is connected with more than one conductive plates.

11. A medium and low frequency electro-stimulated massaging bra comprising:
at least one conductive plate having a plurality of conductive buttons; and
an electro-stimulating controller capable of being connected with said conductive plate by buckling up a corresponding set of conductive buttons with said conductive buttons of said conductive plate;
said conductive plates being combined with said electro-stimulating controller and inserted into an ordinary bra.

12. The medium and low frequency electro-stimulated massaging bra of claim 1, wherein said conductive strips are made of electrically conductive cloth.

13. The medium and low frequency electro-stimulated massaging bra of claim 11, wherein said conductive strips are made of electrically conductive cloth.

14. The medium and low frequency electro-stimulated massaging bra of claim 1, wherein said electro-stimulating controller is placed outside said bra and connected by a wire to said conductive buttons of said bra; said wire has two connecting terminals respectively connected with said conductive buttons of said bra and said conductive buttons of said electro-stimulating controller.
